(19) United States
(12) Patent Application Publication
(10) Pub. No.: US 2005/0049443 A1
(43) Pub. Date: Mar. 3, 2005

(54) Method for producing 1,1,1,3-tetrachloropropane and other haloalkanes using a copper catalyst

(75) Inventor: Richard L. Wilson, Mulvane, KS (US)

Correspondence Address: ARMSTRONG, KRATZ, QUINTOS,;HANSON & BROOKS, LLP, Suite 220, 502 Washington Avenue, Towson, MD, 21204, US

(73) Assignee: VULCAN CHEMICALS A BUSINESS GROUP OF VULCAN MATERIALS COMPANY

(21) Appl. No.: 10/650,654

(22) Filed: Aug. 28, 2003

(51) Int. Cl. C07C 17/02

(52) U.S. Cl. 570/216

(57) ABSTRACT

A process is provided for the manufacture of a haloalkane by the reaction of carbon tetrachloride with an olefin in the presence of dissolved catalytic copper compounds and a cosolvent. A fraction of the catalyst and co-catalyst is recycled to the reactor. In a preferred application, the olefin is ethene and the desired haloalkane is 1,1,1,3-tetrachloropropane, or the olefin is vinyl chloride and the haloalkane is 1,1,1,3,3-pentachloropropane, or the olefin is 1,1-dichloroethene and the haloalkane is 1,1,1,3,3,3-hexachloropropane, or the olefin is 2-chloropropene and the haloalkane product is 1,1,1,3,3-pentachlorobutane. Other preferred implementations provide an efficient continuous process.

### FIELD OF THE INVENTION

The herein disclosed invention finds applicability in the field of haloalkane production.

### BACKGROUND OF THE INVENTION

1,1,1,3-Tetrachloropropane (HCC250fb) is useful as a feedstock to make 1-chloro-3,3,3-tri-fluoropropane (HCFC-253fb), which has been touted as a cleaning solvent that does not contribute substantially to ozone depletion and global warming. 1,1,1,3-tetrachloropropane is also useful as a feedstock to produce 3,3,3-trifluoropropene (HFC-1243zf), which is useful for the production of silicones and agricultural chemicals.

Prior Art Background

GB 971324 (Dow Corning) describes a method for producing 1,1,1,3-tetrachloropropane by the reaction of carbon tetrachloride with ethylene in the presence of di-tertiary-butyl peroxide catalyst at 50-150 psig at a temperature of 100° C. to 140° C. and at an ethylene pressure of from 3.5 to 10.6 Kg/sq. cm.

U.S. Pat. No. 3,651,019 (Asscher) describes a method for the addition of carbon tetrachloride to olefins in a substantially homogeneous reaction medium consisting of an inert organic solvent having iron or copper compounds as the sole catalyst therein. The reaction involves reacting carbon tetrachloride with the olefin in a ratio of 0.05 to 20 moles of carbon tetrachlorides per mole of olefin and is carried out at a temperature of 20 to 300° C. with a dissolved copper or iron compound in a reduced valence state (cuprous or ferrous ions), at a pressure of 1-300 atmospheres and a reaction time of 10 minutes to 48 hours. 1,1,1,3-tetrachloropropane and 1,1,1,5-tetrachloropentane are produced, as well as 1,1,1,3-tetrachloro-3-methylbutane, 1,1,1,3,3-pentachloropropane and 1,1,1,3,3,3-hexachloropropane.

U.S. Pat. No. 5,792,893 (Wilson) describes a process for the preparation of 1,1,1,3,3,3-hexachloropropane by reacting tetrachloromethane with 1,1-dichloroethene using a copper catalyst and alkanenitriles as a solvent. The solvent may be C3 to C5 alkanenitrile. The reaction temperature is 80° to 150° C. with 110° to 150° being preferred.

The prior art does not disclose the inventive process of this invention and in view of the prior art cited, an economical method that provides a high yield of the desired 1, 1,1,3-tetrachloropropane product is still needed.

### OBJECTS OF INVENTION

An object of this invention is to produce haloalkane products in an efficient manner.

A further important object of this invention is to produce 1,1,1,3-tetrachloropropane in an economic and efficient manner.

These and other objects of the invention will become apparent from reading the specification taken in conjunction with the enclosed drawings.

### SUMMARY OF THE INVENTION

The invention provides a continuous or batch process for the manufacture of haloalkanes, in which carbon tetrachloride and an olefin, such as ethene, propene, butene, hexene, heptene, octene, vinyl chloride, 1,1-dichloroethene, 2-chloropropene, 2-chlorobutene, etc., are reacted in the presence of a catalyst mixture comprising a substantially dissolved copper compound and an organonitrile co-catalyst under conditions effective to produce an adduct of carbon tetrachloride and the olefin. The organonitrile co-catalyst is of such nature as to distill overhead at a lower temperature than the desired haloalkane product. The reaction mixture is distilled to produce an overhead mixture containing unconverted reactants and the co-catalyst and a bottoms mixture containing precipitated copper catalyst compounds and the desired haloalkane product. After full or partial separation of the bottoms liquid from the solid, the solids are redissolved in the recovered co-catalyst and sent back to the reactor. The substantially liquid haloalkane product is purified by conventional means to produce a purified haloalkane product. Other embodiments of this invention provide for continuous implementations of this basic design.

### DESCRIPTION OF THE INVENTION

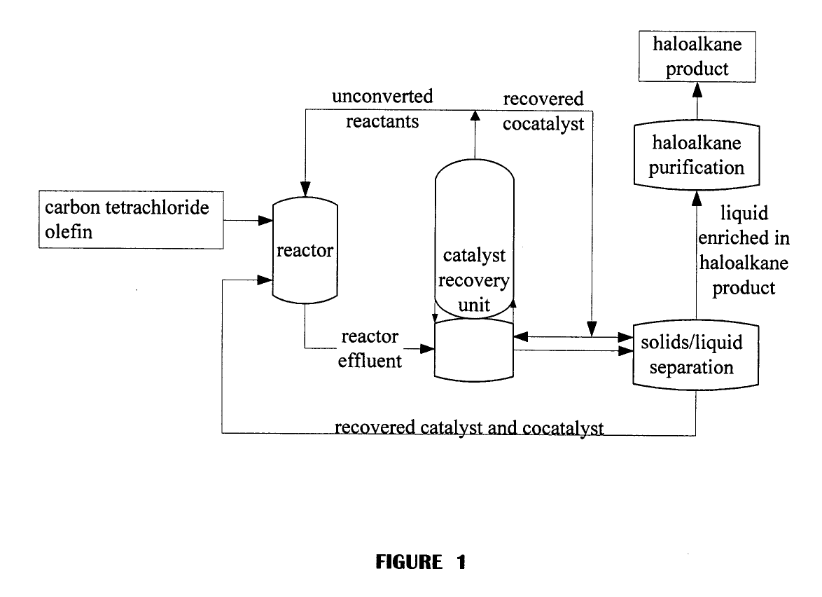

In a first implementation, shown in FIG. 1, the invention provides a process for the production of a haloalkane, in which carbon tetrachloride and an olefin are reacted in the presence of dissolved copper catalysts and an organonitrile co-catalyst under conditions effective to produce the desired haloalkane product. Here, the organonitrile co-catalyst is of such nature as to distill overhead at a lower temperature than the desired haloalkane product. The reactor effluent, which may be a continuous or a periodic stream, is distilled in a catalyst recovery unit to partially remove the co-catalyst and unconverted reactants overhead, which results in the precipitation of the copper catalyst. The catalyst is withdrawn from the catalyst recovery unit as slurry in the haloalkane product. The slurry is separated by conventional means, yielding a substantially liquid haloalkane product, and a solid catalyst or slurry enriched in solid catalyst. Such conventional means of separation include decantation, filtration, hydrocycloning, centrifugation, etc. The clarified liquid haloalkane product may be further purified by any conventional means. The overhead from the catalyst recovery unit may optionally be further separated by conventional means into continuous or periodic streams enriched in unconverted reactants and co-catalyst, respectively. The solid catalyst is then redissolved in purified co-catalyst or in recovered co-catalyst mixed with unconverted reactants, and returned to the reactor.

In an preferred mode based on the first implementation, the olefin is ethene and the desired haloalkane is 1,1,1,3-tetrachloropropane, or the olefin is vinyl chloride and the haloalkane is 1,1,1,3,3-pentachloropropane, or the olefin is 1,1-dichloroethene and the haloalkane is 1,1,1,3,3,3-hexachloropropane, or the olefin is 2-chloropropene and the haloalkane product is 1,1,1,3,3-pentachlorobutane. The organonitrile co-catalyst may be acetonitrile, propanenitrile, butanenitrile, 2-methylpropanenitrile, pentanenitrile, or 3-methoxypropanenitrile. The copper catalyst components are present in an undetermined form, but may be originally added to the system in the form of copper(I) chloride, which is the catalyst precursor.

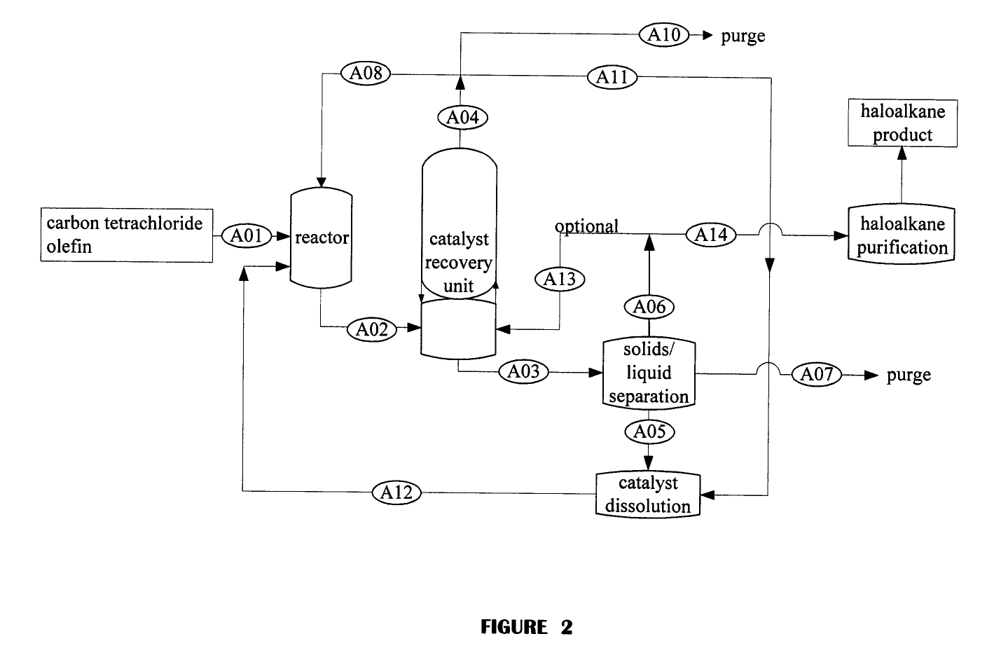

A second implementation of the invention (FIG. 2) provides a continuous process for the production of a haloalkane. Carbon tetrachloride and an olefin (continuous feed streams here labeled A01, which however may be fed to the reactor separately or combined) are reacted in the presence of dissolved copper catalysts and an organonitrile co-catalyst under conditions effective to produce a continuous stream of reactor effluent A02 containing the desired haloalkane product. Here, the organonitrile co-catalyst is of such nature as to distill overhead at a lower temperature than the desired haloalkane product. The reactor effluent A02 is continuously distilled in a catalyst recovery unit, which comprises a distillation column together with equipment that enables the continuous removal of solids. Slurry A03 of catalyst in a fluid enriched in the haloalkane product exits the catalyst recovery unit, and then enters a unit designed to separate the catalyst solids from the liquid. The stream A05 in the figure (FIG. 2) represents either catalyst solids, or slurry that is enriched in catalyst solids. The substantially continuous stream A06 represents a liquid stream that is enriched in haloalkane product. The solids/liquid separation may be achieved by any conventional means known to the art, which include filtration, centrifugation, hydrocycloning, settling tanks, etc. The overhead A04 from the catalyst recovery unit may be substantially pure co-catalyst or substantially a mixture of co-catalyst with unconverted reactants. This stream may optionally be split into two or three continuous streams. Optional stream A08 returns co-catalyst and unconverted reactants to the reactor. Optional purge stream A10 rids the system of undesired low-boiling components. Stream A11 transfers recovered co-catalyst to a unit operation that redissolves the recovered catalyst A05, producing a substantially liquid, substantially continuous stream A12 that returns catalyst and co-catalyst to the reactor. The actual dissolution operation may be periodic or continuous. A purge stream A07, which may be periodic or continuous, rids the system of unwanted high-boiling components. The liquid haloalkane product stream A06 may be further purified by conventional means, which include distillation, drying, contacting with absorptive substances, etc. Any catalyst and co-catalyst that are purged from the system via A07 or A10 may be replaced with fresh organonitrile and a copper compound that is soluble in the organonitrile, not shown on the diagram.

In a preferred mode based on the second implementation, the olefin is ethene and the desired haloalkane is 1,1,1,3-tetrachloropropane, or the olefin is vinyl chloride and the haloalkane is 1,1,1,3,3-pentachloropropane, or the olefin is 1,1-dichloroethene and the haloalkane is 1,1,1,3,3,3-hexachloropropane, or the olefin is 2-chloropropene and the haloalkane product is 1,1,1,3,3 -pentachlorobutane. The organonitrile co-catalyst is acetonitrile, propanenitrile, or butanenitrile, or 2-methylpropanenitrile, or pentanenitrile, or 3-methoxypropanenitrile. The copper catalyst components are present in an undetermined form, but are originally added to the system in the form of copper(I) chloride, which is the catalyst precursor.

Another preferred mode based on the second implementation (FIG. 2) employs for the first catalyst recovery unit a packed distillation column located above a hydrocyclone, so that solids produced during the distillation are continuously swept out the bottom of the hydrocyclone by recirculating liquid which has been at least partially cleared of solids. For instance, the stream A06 may be split into two streams A13 and A14, respectively. A13 is returned to the hydrocyclone to enhance the sweeping action, while A14 carries the desired haloalkane product to the haloalkane purification section.

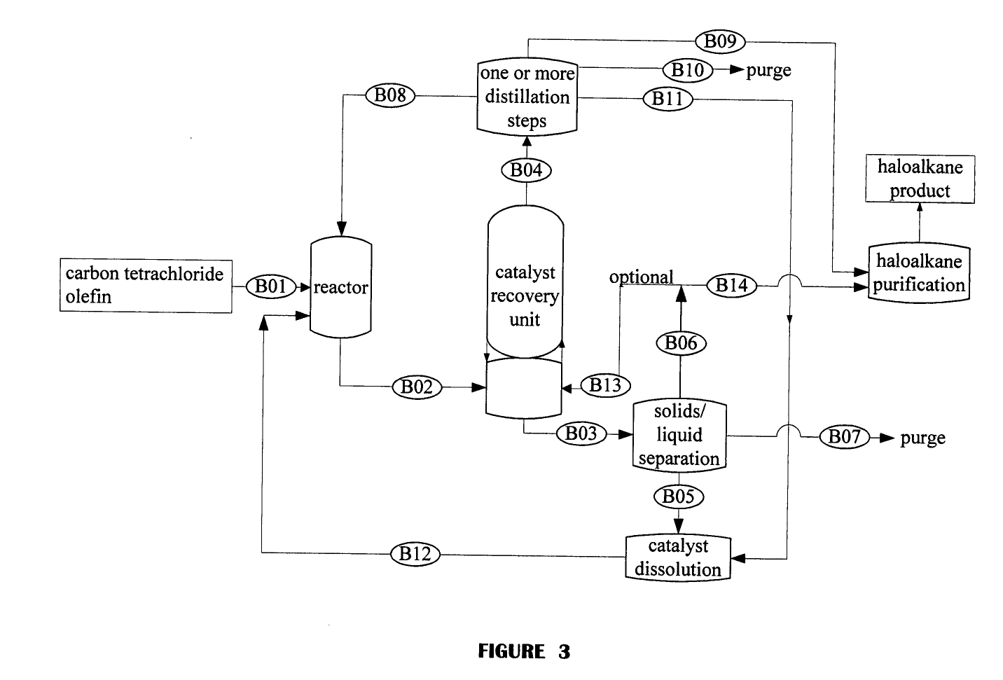

A third implementation of the invention (FIG. 3) provides a continuous process for the production of a haloalkane, in which carbon tetrachloride and an olefin (continuous feed streams here labeled B01, which however may be fed to the reactor separately or combined) are reacted in the presence of dissolved copper catalysts and an organonitrile co-catalyst under conditions effective to produce a continuous stream of reactor effluent B02 containing the desired haloalkane product. Here, the organonitrile co-catalyst is of such nature as to distill overhead at a lower temperature than the desired haloalkane product. The reactor effluent B02 is continuously distilled in a catalyst recovery unit, which comprises a distillation column together with equipment that enables the continuous removal of solids. Slurry B03 of catalyst in a fluid enriched in the haloalkane product exits the catalyst recovery unit, and then enters a unit designed to separate the catalyst solids from the liquid. The stream B05 in the figure represents either catalyst solids, or slurry that is enriched in catalyst solids. The substantially continuous stream B06 represents a liquid stream that is enriched in haloalkane product. The solids/liquid separation may be achieved by any conventional means known to the art, which include filtration, centrifugation, hydrocycloning, settling tanks, etc. The overhead B04 from the catalyst recovery unit may be substantially pure co-catalyst or substantially a mixture of co-catalyst with unconverted reactants, or substantially a mixture of co-catalyst, unconverted reactants, and the desired haloalkane product. This stream may be separated into several continuous streams by a distillation unit or several distillation units. Optional stream B08 returns co-catalyst and/or unconverted reactants to the reactor. Optional stream B09 transfers a mixture that is enriched in the desired haloalkane product to the haloalkane purification section. Optional purge stream B10 rids the system of undesired low-boiling components. Stream B11 transfers recovered co-catalyst to a unit operation that redissolves the recovered catalyst B05, producing a substantially liquid, substantially continuous stream B12 that returns catalyst and co-catalyst to the reactor. The actual dissolution operation may be periodic or continuous. A purge stream B07, which may be periodic or continuous, rids the system of unwanted low-boiling components. The liquid haloalkane product stream B06 may be further purified by conventional means, which include distillation, drying, contacting with absorptive substances, etc. Any catalyst and co-catalyst that are purged from the system via B07 or B10 may be replaced with fresh organo-nitrile and a copper compound that is soluble in the organonitrile, not shown on the diagram.

In a preferred mode based on the third implementation (FIG. 3), the olefin is ethene and the desired haloalkane is 1,1,1,3-tetrachloropropane, or the olefin is vinyl chloride and the haloalkane is 1,1,1,3,3-pentachloropropane, or the olefin is 1,1-dichloroethene and the haloalkane is 1,1,1,3,3,3-hexachloropropane, or the olefin is 2-chloropropene and the haloalkane product is 1,1,1,3,3-pentachlorobutane. The organonitrile co-catalyst is acetonitrile, propanenitrile, or butanenitrile, or 2-methylpropanenitrile, or pentanenitrile, or 3-methoxypropanenitrile. The copper catalyst components are present in an undetermined form, but are originally added to the system in the form of copper(I) chloride, which is the catalyst precursor.

Another preferred mode based on the third implementation (FIG. 3), employs for the first catalyst recovery unit a packed distillation column located above a hydrocyclone, so that solids produced during the distillation are continuously swept out the bottom of the hydrocyclone by re-circulating liquid which has been at least partially cleared of solids. For instance, the stream B06 may be split into two streams B13 and B14. B13 is returned to the hydrocyclone to enhance the sweeping action, while B14 carries the desired haloalkane product to the haloalkane purification section.

The reaction conditions are selected to be effective for the addition of carbon tetrachloride with the olefin to make the desired adduct. In general, the reaction temperature will be from 80 to 170 C, the pressure will be from 10 to 500 psig, and the molar feed ratio of carbon tetrachloride to olefin will be from 1.05 to 2.0. The reaction mixture is substantially anhydrous, containing from about 5 to 1500 ppm water. The organonitrile is very slowly consumed, producing unwanted byproducts that must be purged from the system. The organonitrile must therefore be replaced, either continuously or periodically. Likewise, traces of unwanted high-boiling or non-boiling substances will accumulate in the system unless there is a high-boiler purge, which can be periodic or continuous. Copper catalyst that is purged by this route must be replaced with fresh copper(I) chloride. Some or all of the distillation operations will be preferably done at reduced pressure, since the desired halocarbon products tend to be unstable at high temperatures. The presence of iron chlorides or other strong Lewis acids should be avoided in solutions that contain the desired halocarbon product, as these catalyze unwanted dehydrochlorination reactions. Certain compounds may be added to counteract such Lewis acid activity, such as certain amines, 1-hydroxy-4-methoxy-phenol, etc.

Mixtures of copper compounds with organonitriles are quite corrosive to most metals. Accordingly, equipment that is wetted by such solutions must be especially designed to resist such attack. In general, such equipment will be of glass-, teflon-, lead-, or tantalum-lined, or the like. This consideration provides some incentive to consider taking a portion of the desired haloalkane product overhead in the catalyst recovery unit, if such is necessary to assure that no significant co-catalyst remains in the bottom liquid. The copper catalyst tends not to be soluble in haloalkanes without the presence of co-catalyst. Undissolved copper catalyst is much less corrosive to metals than is dissolved copper catalyst. This design could help to minimize corrosion of downstream metallic equipment—for example, metallic solids/liquid separation equipment—in a cost-effective way.

Examples of processes for carrying out the invention:

### EXAMPLE 1

The herein disclosed invention encompasses a process for the manufacture of a haloalkane comprising the steps:
a) contacting carbon tetrachloride with an olefin in a reactor in the presence of a substantially dissolved catalytic copper compound and an organonitrile co-catalyst, wherein the co-catalyst is selected to be capable of distilling at a lower temperature than the desired haloalkane product, under conditions effective to produce a reaction mixture that contains a haloalkane adduct of carbon tetrachloride with the olefin,
b) distilling the reaction mixture in a catalyst recovery unit to produce an overhead stream or overhead streams containing unconverted reactants and recovered organonitrile co-catalyst and a bottoms stream comprising a slurry of precipitated copper catalyst components in a fluid that is enriched in the desired haloalkane product,
c) separating the precipitated copper catalyst components from the fluid that is enriched in the desired haloalkane product and purifying the fluid by conventional means to yield purified haloalkane product,
d) dissolving the precipitated copper catalyst components in the recovered organonitrile co-catalyst to produce a solution of copper catalyst in a liquid containing the organonitrile co-catalyst,
e) returning a portion of the catalyst solution copper catalyst in organonitrile co-catalyst from d) to the reactor. In the process, the olefin can be ethene or vinyl chloride and the desired haloalkane can be 1,1,1,3-tetrachloropropane or 1,1,1,3,3-pentachloropropane. Alternatively the olefin can be 1,1-dichloroethene and the haloalkane can be 1,1,1,3,3,3-hexachloropropane, or 2-chloropropene and 1,1,1,3,3-pentachlorobutane. In the process, the organonitrile co-catalyst can be acetonitrile, propanenitrile, butanenitrile, 2-methylpropanenitrile, pentanenitrile, or 3-methoxypropanenitrile. The copper catalyst components are present during reaction in an undetermined form, but may be originally added to the system in the form of copper(I) chloride, which is the catalyst precursor. In the process, a portion of the unconverted reactants from step b) is returned to the reactor.

### EXAMPLE 2

More specifically the process for the manufacture of 1,1,1,3-tetrachloro-propane comprises the steps:
a) contacting carbon tetrachloride with ethene in a reactor in the presence of substantially dissolved copper catalyst components, wherein the copper catalyst components were originally added to the process in the form of cuprous chloride, and n-butyronitrile co-catalyst, under conditions effective to produce a reaction mixture that contains 1,1,1,3-tetrachloropropane, wherein the reactor preferably operates at 100-180° C. and at 80-400 psig, with liquid residence time of 0.2-200 hours, and with liquid n-butyronitrile concentration from 10-50 wt %,
b) distilling the reaction mixture in a catalyst recovery unit to produce an overhead stream or overhead streams containing unconverted reactants and recovered co-catalyst, and a bottoms stream comprising a slurry of precipitated copper catalyst components in a fluid that is enriched in 1,1,1,3-tetrachloropropane, wherein the distillation is preferably done at a temperature of from 30-160° C., and at pressure 5-840 torr,
c) separating the precipitated copper catalyst components from the bottoms slurry from b) and purifying the liquid by conventional means, such as distillation, to yield purified 1,1,1,3-tetrachloropropane,
d) dissolving the precipitated copper catalyst components from c) in the recovered n-butyronitrile co-catalyst from b) to produce a solution of copper catalyst components in a liquid containing the co-catalyst, and e) returning a portion of the catalyst solution from d) to the reactor.

Still more specifically, the reactor operates at 120-160° C. and at 100-300 psig, with liquid residence time of 1-50 hours, with the liquid n-butyronitrile concentration of 20-40 wt %, and in step b) the distillation is done at a temperature of from 88-112° C. and at a pressure of 85-204 torr. In a preferred embodiment of the invention, the reactor operates at 138° C. and at 250 psig, with a liquid residence time of about 5 hours if a batch reaction is employed or 11 hours if a continuous reaction is employed, and with the liquid n-butyronitrile concentration of about 30 wt % and in step b) the distillation is done at a temperature of about 104° C. at a pressure of 154 torr.

### EXAMPLE 3

In an alternative process for the manufacture of a haloalkane, the process is carried out by the steps of:
a) contacting carbon tetrachloride with an olefin in a reactor in the presence of a substantially dissolved catalytic copper compound and an organonitrile co-catalyst, wherein the co-catalyst is selected to be capable of distilling at a lower temperature than the desired haloalkane product, under conditions effective to produce a continuous reactor effluent stream that contains a haloalkane adduct of carbon tetrachloride with the olefin,
b) continuously distilling the reactor effluent stream in a catalyst recovery unit to produce an overhead stream or overhead streams containing unconverted reactants and recovered co-catalyst and a bottoms stream comprising a slurry of precipitated copper catalyst components in a fluid that is enriched in the desired haloalkane product,
c) separating the solid copper catalyst components from the fluid in a solids/liquid separation unit to produce a continuous, substantially liquid stream containing the desired haloalkane product and a solid product that contains the solid copper catalyst components or a slurry that is enriched in the solid catalyst components,
d) purifying by distillation the liquid stream containing the desired haloalkane product from c) to produce a purified haloalkane product,
e) dissolving the solid copper catalyst components from c) in the recovered co-catalyst stream from b) to produce a solution of copper catalyst components in a liquid containing the co-catalyst, and
f) continuously feeding a portion of the liquid catalyst/co-catalyst solution from e) into the reactor.

In the process a portion of the unconverted reactants from step b) is returned to the reactor. The catalyst recovery unit comprises a distillation column located above a hydrocyclone unit wherein the solids formed during distillation are continuously swept from the hydrocyclone using a liquid stream taken from the solids/liquid separation unit of step c). In a preferred method for the manufacture of 1,1,1,3-tetrachloropropane of the above process step a) the reactor preferably operates at 100-180° C. and at 80-400 psig, with liquid residence time 0.2-200 hours, and with liquid n-butyronitrile concentration from 10-50 wt %. In step b) the desired haloalkane is 1,1,1,3-tetrachloropropane, and the distillation is preferably done at a temperature of from 30-160° C., and at pressure of 5-840 torr. In step c) the substantially liquid stream contains 1,1,1,3-tetrachloropropane product and either a substantially solid product that contains the solid copper catalyst components or a slurry that is enriched in the solid catalyst components, wherein the solids/liquid separation device is a filter, a centrifuge or a hydrocyclone, and is preferably a hydrocyclone, purifying the substantially liquid stream containing 1,1,1,3-tetrachloropropane product from c) to produce a purified 1,1,1,3-tetrachloropropane product, wherein the purification method is substantially distillation, and dissolving the solid copper catalyst components from c) in the recovered n-butyronitrile co-catalyst stream from b) to produce a solution of copper catalyst components in a liquid containing the co-catalyst, and continuously feeding a portion of a liquid catalyst/co-catalyst solution from e) into the reactor. In a more specific process, the reactor operates at 120-160° C. and at 100-300 psig with liquid residence time of 1-50 hours and with a liquid n-butyronitrile concentration of 20-40 wt % and in step b) the distillation is done at a temperature of 88-112° C. and at a pressure of 85-204 torr and in a still more specific process the reactor operates at a temperature of 138° C. and at a pressure of 250 psig with liquid residence time of about 11 hours and with a liquid n-butyronitrile concentration of 30 wt % and in step b) the distillation is done at a temperature of 104° C. and at a pressure of 154 torr.

### EXAMPLE 4

The invention also involves a process for the manufacture of a haloalkane comprising:
a) contacting carbon tetrachloride with an olefin in a reactor in the presence of a substantially dissolved catalytic copper compound and an organonitrile co-catalyst, wherein the co-catalyst is selected to be capable of distilling at a lower temperature than the desired haloalkane product, under conditions effective to produce a continuous reactor effluent stream that contains a haloalkane adduct of carbon tetrachloride with the olefin,
b) continuously distilling the reactor effluent stream in a catalyst recovery unit to produce an overhead stream containing unconverted reactants, recovered co-catalyst, and the desired haloalkane product, and a bottoms stream comprising a slurry of precipitated copper catalyst components in a fluid that is enriched in the desired haloalkane product,
c) continuously distilling the overhead stream from b) to produce streams that are enriched in unconverted reactants and co-catalyst, respectively, and optionally also to produce a stream that is enriched in the desired haloalkane product,
d) separating the solid copper catalyst components from the bottom fluid of step b) in a solids/liquid separation unit to produce a continuous, substantially liquid stream containing the desired haloalkane product and a solid product that contains the copper catalyst components or a slurry that is enriched in the solid catalyst components,
e) optionally combining the stream that is enriched in the desired haloalkane product from step c) with the liquid haloalkane stream from step d),
f) purifying by distillation the liquid stream containing the desired haloalkane product from d) or e) to produce a purified haloalkane product,
g) dissolving the solid copper catalyst components from d) in the enriched co-catalyst stream from c) to produce a solution of copper catalyst components in a liquid containing the co-catalyst, and
h) continuously feeding a portion of the liquid catalyst/co-catalyst solution from g) into the reactor

### EXAMPLE 5

A still further process for the manufacture of 1,1,1,3-tetrachloropropane comprises the steps of:
a) contacting carbon tetrachloride with ethene in a reactor in the presence of substantially dissolved cuprous chloride catalyst and n-butyronitrile co-catalyst, under conditions effective to produce a continuous reactor effluent stream that contains 1,1,1,3-tetrachloropropane, wherein the reactor operates at 100-180° C. and at 80-400 psig with liquid residence time 0.2-200 hours, and with liquid n-butyronitrile concentration from 10-50 wt %,
b) continuously distilling the reactor effluent stream in a catalyst recovery unit to produce an overhead stream containing unconverted reactants and recovered co-catalyst and less than five percent of the 1,1,1,3-tetrachloropropane contained in the reactor effluent, and a bottoms stream comprising a slurry of precipitated copper catalyst components in a fluid that is enriched in 1,1,1,3-tetrachloropropane product, wherein the distillation is done at a temperature of from 30-160° C., and at pressure 5-840 torr,
c) continuously distilling the overhead stream from b) to produce streams that are enriched in unconverted reactants and co-catalyst, respectively, wherein the co-catalyst stream contains less that five percent of the 1,1,1,3-tetrachloropropane contained in the reactor effluent stream, and
d) separating the solid copper catalyst components from the bottom fluid of step b) in a solids/liquid separation unit to produce a continuous, substantially liquid stream containing greater than 80% of the 1,1,1,3-tetrachloropropane that is contained in the reactor effluent, and either a solid product that contains the copper catalyst components or a slurry that is enriched in the solid catalyst components, wherein the solids/liquid separation device is a filter, a centrifuge or a hydrocyclone, and is preferably a hydrocyclone, and
e) distilling the liquid stream containing the 1,1,1,3-tetrachloropropane product from d) to produce a purified 1,1,1,3-tetrachloropropane product,
f) dissolving the solid copper catalyst components from d) in the enriched co-catalyst stream from c) to produce a solution of copper catalyst components in a liquid containing the co-catalyst, and
g) continuously feeding a portion of the liquid catalyst/co-catalyst solution from f) into the reactor. In a preferred embodiment, the reactor operates at 120-160° C. and at 100-300 psig with a liquid residence time of 1-50 hours and with a liquid n-butyronitrile concentration of from 20-40 wt % and in step b) the distillation is done at a temperature of 88-112° C. and pressure of 85-201 torr. In a most preferred embodiment, the reactor operates at a temperature of 138° C. and at a pressure of 250 psig with a liquid residence time of 11 hours and with a liquid n-butyronitrile concentration of from 30 wt % and in step b) the distillation is done at a temperature of 104° C. and pressure of 154 torr.

Obviously, many modifications may be made without departing from the basic spirit of the present invention. Accordingly, it will be appreciated by those skilled in the art that within the scope of the appended claims, the invention may be practiced other than has been specifically described herein.

1. A process for the manufacture of a haloalkane comprising the steps of:
a) contacting carbon tetrachloride with an olefin in a reactor in the presence of a substantially dissolved catalytic copper compound and an organonitrile co-catalyst, wherein the co-catalyst is selected to be capable of distilling at a lower temperature than the desired haloalkane product, under conditions effective to produce a reaction mixture that contains a haloalkane adduct of carbon tetrachloride with the olefin,
b) distilling the reaction mixture in a catalyst recovery unit to produce an overhead stream or overhead streams containing unconverted reactants and recovered organonitrile co-catalyst and a bottoms stream comprising a slurry of precipitated copper catalyst components in a fluid that is enriched in the desired haloalkane product,
c) separating the precipitated copper catalyst components of step b) from the fluid that is enriched in the desired haloalkane product and purifying by distillation the fluid to yield purified haloalkane product,
d) dissolving the precipitated copper catalyst components of step c) in the recovered organonitrile co-catalyst of step b) to produce a solution of copper catalyst in a liquid containing the organonitrile co-catalyst and
e) returning a portion of the copper catalyst solution in organonitrile co-catalyst from step d) to the reactor.

2. The process of claim 1 wherein the olefin is ethene and the desired haloalkane is 1,1,1,3-tetrachloropropane or the olefin is vinyl chloride and the haloalkane is 1,1,1,3,3-pentachloropropane, or the olefin is 1,1-dichloroethene and the haloalkane is 1,1,1,3,3,3-hexachloropropane, or the olefin is 2-chloropropene and the haloalkane product is 1,1,1,3,3-pentachlorobutane.

3. The process of claim 1 wherein the organonitrile co-catalyst is acetonitrile, propanenitrile, butanenitrile, 2-methylpropanenitrile, pentanenitrile, or 3-methoxypropanenitrile.

4. The process of claim 1 wherein the copper catalyst components which are present in an undetermined form during reaction are originally added to the system in the form of copper(I) chloride, which is the catalyst precursor.

5. The process of claim 1 wherein a portion of the unconverted reactants from step b) is returned to the reactor.

6. A process for the manufacture of 1,1,1,3-tetrachloropropane comprising the steps of:
a) contacting carbon tetrachloride with ethene in a reactor in the presence of substantially dissolved copper catalyst components, wherein the copper catalyst components were originally added to the process in the form of cuprous chloride, and n-butyronitrile co-catalyst, under conditions effective to produce a reaction mixture that contains 1,1,1,3-tetrachloropropane, wherein the reactor preferably operates at 100-180° C. and at 80-400 psig, with liquid residence time of 0.2-200 hours, and with liquid n-butyronitrile concentration from 10-50 wt %,
b) distilling the reaction mixture in a catalyst recovery unit to produce an overhead stream or overhead streams containing unconverted reactants and recovered co-catalyst, and a bottoms stream comprising a slurry of precipitated copper catalyst components in a fluid that is enriched in 1,1,1,3-tetrachloropropane, wherein the distillation is preferably done at a temperature of from 30-160° C., and at pressure 5-840 torr,
c) separating the precipitated copper catalyst components from the bottoms slurry from b) and purifying the liquid by conventional means, such as distillation, to yield purified 1,1,1,3-tetrachloropropane,
d) dissolving the precipitated copper catalyst components from c) in the recovered n-butyronitrile co-catalyst from b) to produce a solution of copper catalyst components in a liquid containing the co-catalyst and
e) returning a portion of the catalyst solution from d) to the reactor.

7. The process for manufacture of 1,1,1,3-tetrachloropropane of claim 6 wherein in step a) the reactor operates at 120-160° C. and at 100-300 psig, with liquid residence time of 1-50 hours, with the liquid n-butyronitrile concentration of 20-40 wt %, and in step b) the distillation is done at a temperature of from 88-112° C. and at a pressure of 85-204 torr.

8. The process for manufacture of claim 7 wherein the reactor operates at 138° C and at 250 psig, with a liquid residence time of about 5 hours if a batch reaction is employed or 11 hours if a continuous reaction is employed, and with the liquid n-butyronitrile concentration of about 30 wt % and in step b) the distillation being done at a temperature of about 104° C. and at a pressure of 154 torr.

9. A process for the manufacture of a haloalkane comprising the steps of:
a) contacting carbon tetrachloride with an olefin in a reactor in the presence of a substantially dissolved catalytic copper compound and an organonitrile co-catalyst, wherein the co-catalyst is selected to be capable of distilling at a lower temperature than the desired haloalkane product, under conditions effective to produce a continuous reactor effluent stream that contains a haloalkane adduct of carbon tetrachloride with the olefin,
b) continuously distilling the reactor effluent stream in a catalyst recovery unit to produce an overhead stream or overhead streams containing unconverted reactants and recovered co-catalyst and a bottoms stream comprising a slurry of precipitated copper catalyst components in a fluid that is enriched in the desired haloalkane product,
c) separating the solid copper catalyst components of step b) from the fluid in a solids/liquid separation unit to produce a continuous, substantially liquid stream containing the desired haloalkane product and a solid product that contains the solid copper catalyst components or a slurry enriched in the solid catalyst components,
d) purifying by distillation the liquid stream containing the desired haloalkane product from step c) to produce a purified haloalkane product,
e) dissolving the solid copper catalyst components from step c) in the recovered co-catalyst stream from step b) to produce a solution of copper catalyst components in a liquid containing the co-catalyst and
f) continuously feeding a portion of the liquid catalyst/co-catalyst solution from step e) into the reactor.

10. The process of claim 9 wherein the olefin is ethene and the desired haloalkane is 1,1,1,3-tetrachloropropane or the olefin is vinyl chloride and the haloalkane is 1,1,1,3,3-pentachloropropane, or the olefin is 1,1-dichloroethene and the haloalkane is 1,1,1,3,3,3-hexachloropropane, or the olefin is 2-chloropropene and the haloalkane product is 1,1,1,3,3-pentachlorobutane.

11. The process of claim 9 wherein the organonitrile co-catalyst is acetonitrile, propanenitrile, butanenitrile, 2-methylpropanenitrile, pentanenitrile, or 3-methoxypropanenitrile.

12. The process of claim 9 wherein the copper catalyst components are originally added to the system in the form of copper(I) chloride, which is the catalyst precursor.

13. The process of claim 9 wherein a portion of the unconverted reactants from step b) is returned to the reactor.

14. The process of claim 9 wherein the catalyst recovery unit comprises a distillation column located above a hydrocyclone unit wherein the solids formed during distillation are continuously swept from the hydrocyclone using a liquid stream taken from the solids/liquid separation unit of step c).

15. A process for manufacture of 1,1,1,3-tetrachloropropane comprising the steps of:
a) contacting carbon tetrachloride with ethene in a reactor in the presence of substantially dissolved cuprous chloride catalyst and n-butyronitrile cocatalyst, under conditions effective to produce a continuous reactor effluent stream that contains 1,1,1,3-tetrachloropropane, wherein the reactor operates at 100-180° C. and at 80-400 psig, with liquid residence time 0.2-200 hours, and with liquid n-butyronitrile concentration from 10-50 wt %,
b) continuously distilling the reactor effluent stream in a catalyst recovery unit to produce an overhead stream or overhead streams containing unconverted reactants and recovered cocatalyst and a bottoms stream comprising a slurry of precipitated copper catalyst components in a fluid that is enriched in 1,1,1,3-tetrachloropropane product, wherein the distillation is done at a temperature from 30-160° C., and at pressure of 5-840 torr,
c) separating the solid copper catalyst components from the fluid in a solids/liquid separation unit to produce a continuous, substantially liquid stream containing 1,1,1,3-tetrachloropropane product and either a substantially solid product that contains the solid copper catalyst components or a slurry that is enriched in the solid catalyst components, wherein the solids/liquid separation device is a filter, a centrifuge or a hydrocyclone, and is preferably a hydrocyclone,
d) purifying by distillation the substantially liquid stream containing 1,1,1,3-tetrachloropropane product from c) to produce a purified 1,1,1,3-tetrachloropropane product,
e) dissolving the solid copper catalyst components from c) in the recovered n-butyronitrile co-catalyst stream from b) to produce a solution of copper catalyst components in a liquid containing the co-catalyst, and
f) continuously feeding a portion of a liquid catalyst/co-catalyst solution from e) into the reactor.

16. The process for the manufacture of 1,1,1,3-tetrachloropropane of claim 15 wherein in step a) the reactor operates at 120-160° C. and at 100-300 psig with liquid residence time of 1-50 hours and with a liquid n-butyronitrile concentration of 20-40 wt % and in step b) the distillation is done at a temperature of 88-112° C. and at a pressure of 85-204 torr.

17. The process for the manufacture of 1,1,1,3-tetrachloropropane of claim 16 wherein in step a) the reactor operates at a temperature of 138° C. and at a pressure of 250 psig with liquid residence time of about 11 hours and with a liquid n-butyronitrile concentration of 30 wt % and in step b) the distillation is done at a temperature of 104° C. and at a pressure of 154 torr.

18. A process for the manufacture of a haloalkane comprising the steps of:
a) contacting carbon tetrachloride with an olefin in a reactor in the presence of a substantially dissolved catalytic copper compound and an organonitrile co-catalyst, wherein the co-catalyst is selected to be capable of distilling at a lower temperature than the desired haloalkane product, under conditions effective to produce a continuous reactor effluent stream that contains a haloalkane adduct of carbon tetrachloride with the olefin,
b) continuously distilling the reactor effluent stream in a catalyst recovery unit to produce an overhead stream containing unconverted reactants, recovered co-catalyst, and the desired haloalkane product, and a bottoms stream comprising a slurry of precipitated copper catalyst components in a fluid that is enriched in the desired haloalkane product,
c) continuously distilling the overhead stream from b) to produce streams that are enriched in unconverted reactants and co-catalyst, respectively, and optionally also to produce a stream that is enriched in the desired haloalkane product,
d) separating the solid copper catalyst components from the bottom fluid of step b) in a solids/liquid separation unit to produce a continuous, substantially liquid stream containing the desired haloalkane product and a solid product that contains the copper catalyst components or a slurry that is enriched in the solid catalyst components,
e) optionally combining the stream that is enriched in the desired haloalkane product from step c) with the liquid haloalkane stream from step d),
f) purifying by distillation the liquid stream containing the desired haloalkane product from d) or e) to produce a purified haloalkane product,
g) dissolving the solid copper catalyst components from d) in the enriched co-catalyst stream from c) to produce a solution of copper catalyst components in a liquid containing the co-catalyst, and
h) continuously feeding a portion of the liquid catalyst/co-catalyst solution from g) into the reactor.

19. The process of claim 18 wherein the olefin is ethene and the desired haloalkane is 1, 1, 1, 3-tetrachloropropane or the olefin is vinyl chloride and the haloalkane is 1,1,1,3,3-pentachloropropane, or the olefin is 1,1-dichloro-ethene and the haloalkane is 1,1,1,3,3,3-hexachloropropane, or the olefin is 2-chloropropene and the haloalkane product is 1,1,1,3,3-pentachlorobutane.

20. The process of claim 18 wherein the organonitrile co-catalyst is acetonitrile, propanenitrile, butanenitrile, 2-methylpropanenitrile, pentanenitrile, or 3-methoxypropanenitrile.

21. The process of claims 18 wherein the copper catalyst components are present in an undetermined form, but may be originally added to the system in the form of copper(I) chloride, which is the catalyst precursor.

22. The process of claim 18 wherein the unconverted reactants from step c) are returned to the reactor.

23. The process of claim 18 wherein the catalyst recovery unit comprises a distillation column located above a hydrocyclone unit wherein the solids formed during distillation are continuously swept from the hydrocyclone using a liquid stream taken from the solids/liquid separation unit of step d).

24. A process for the manufacture of 1,1,1,3-tetrachloropropane comprising the steps of:
a) contacting carbon tetrachloride with ethene in a reactor in the presence of substantially dissolved cuprous chloride catalyst and n-butyronitrile co-catalyst, under conditions effective to produce a continuous reactor effluent stream that contains 1,1,1,3 -tetrachloropropane, wherein the reactor operates at 100-180° C. and at 80-400 psig with liquid residence time 0.2-200 hours, and with liquid n-butyronitrile concentration from 10-50 wt %,
b) continuously distilling the reactor effluent stream in a catalyst recovery unit to produce an overhead stream containing unconverted reactants and recovered co-catalyst and less than five percent of the 1,1,1,3-tetrachloropropane contained in the reactor effluent, and a bottoms stream comprising a slurry of precipitated copper catalyst components in a fluid that is enriched in 1,1,1,3-tetrachloropropane product, wherein the distillation is done at a temperature of from 30-160° C., and at pressure 5-840 torr,
c) continuously distilling the overhead stream from b) to produce streams that are enriched in unconverted reactants and co-catalyst, respectively, wherein the co-catalyst stream contains less that five percent of the 1,1,1,3-tetrachloropropane contained in the reactor effluent stream, and
d) separating the solid copper catalyst components from the bottom fluid of step b) in a solids/liquid separation unit to produce a continuous, substantially liquid stream containing greater than 80% of the 1,1,1,3-tetrachloropropane that is contained in the reactor effluent, and either a solid product that contains the copper catalyst components or a slurry that is enriched in the solid catalyst components, wherein the solids/liquid separation device is a filter or a centrifuge or a hydrocyclone, and is preferably a hydrocyclone, and
e) distilling the liquid stream containing the 1,1,1,3-tetrachloropropane product from d) to produce a purified 1,1,1,3-tetrachloropropane product,
f) dissolving the solid copper catalyst components from d) in the enriched co-catalyst stream from c) to produce a solution of copper catalyst components in a liquid containing the co-catalyst, and
g) continuously feeding a portion of the liquid catalyst/co-catalyst solution from f) into the reactor.

25. The process for the manufacture of 1,1,1,3-tetrachloropropane of claim 24 wherein in step a) the reactor operates at 120-160° C. and at 100-300 psig with a liquid residence time of 1-50 hours and with a liquid n-butyronitrile concentration of from 20-40 wt % and in step b) the distillation is done at a temperature of 88-112° C. and pressure of 85-201 torr.

26. The process for the manufacture of 1,1,1,3-tetrachloropropane of claim 25 wherein in step a) the reactor operates at a temperature of 138° C. and at a pressure of 250 psig with a liquid residence time of 11 hours and with a liquid n-butyronitrile concentration of from 30 wt % and in step b) the distillation is done at a temperature of 104° C. and pressure of 154 torr.
